(19) United States
(12) Patent Application Publication
(10) Pub. No.: US 2026/0002111 A1
(43) Pub. Date: Jan. 1, 2026

(54) TISSUE CULTURE CHIP AND TISSUE CULTURE SYSTEM

(71) Applicant: National Cheng Kung University, Tainan (TW)

(72) Inventors: Chen-Hsun WENG, Tainan (TW); Ping-Han CHUANG, Tainan (TW); Tsung-Lin TSAI, Tainan (TW)

(21) Appl. No.: 18/773,884

(22) Filed: Jul. 16, 2024

(30) Foreign Application Priority Data
Jun. 26, 2024 (TW) 113123728

(51) Int. Cl. C12M 1/34; C12M 1/00; C12M 1/32

(52) U.S. Cl. CPC C12M 41/36; C12M 23/12; C12M 23/26

(57) ABSTRACT

The present invention discloses a tissue culture chip, comprising: a flexible substrate having a bottom surface and a top surface comprising: a sensing part, arranged between the bottom surface and the top surface; a culturing part, arranged on the sensing part, the culturing part comprises a first culturing chamber and a second culturing chamber; and a circulating part, arranged on the sensing part and near the culturing part, and fluid communicated with the culturing part.

### FIELD OF THE INVENTION

The present invention relates to tissue culture, and more precisely, relates to tissue culture chip, tissue culture system and tissue culture method using the same.

### BACKGROUND OF THE INVENTION

All living organisms are composed of cells, therefore taking observation of the cells, such as via an in vitro cell culture, is one of the most important aspects of present biotechnology to obtain better understandings of living organisms, including exploring nature of the cells, effects of substances on the cells.

As well acknowledged, some cell culture methods include implanting cells into a cell culture plate, adding culture medium into the cell culture plate, and then placing the cell culture plate into the incubator. However, cells cultured by the aforementioned culture method are somehow different from cells grow in vivo. Key factors may include differences of oxygen partial pressure, culture medium, and interaction with the environment. As a result, a drug test based on the cell culture loses precision or accuracy, which brings negative impacts on drug development industry.

In addition, a tissue culture requires further improvement in terms of 3D culture, testing or applications.

### SUMMARY OF THE INVENTION

To overcome the defects in the prior arts, a tissue culture chip is provided by the present invention, comprising a flexible substrate, having a bottom surface and a top surface, including: a sensing part, arranged between the bottom surface and the top surface; a culturing part, arranged on the sensing part, comprising a first culture well and a second culture well; and a circulating part, arranged on the sensing part that near the culturing part.

In one embodiment, the tissue culture chip of claim 1, wherein a Young's modulus of the flexible substrate ranges between 800-1800 (megapascal, Mpa) when a radius of curvature of the flexible substrate ranges between 50-100 (millimeter, mm), wherein the radius of curvature of the flexible substrate is measured when the flexible substrate is being bent to enable both end of the flexible substrate getting closer toward the bottom surface or the top surface.

In one embodiment, the flexible substrate is made of one or more materials of the following: polydimethylsiloxane, polyimide, polyethylene, poly (methyl methacrylate), polycarbonate, polyethylene terephthalate, polyethylene naphthalate, polytetrafluoroethylene, polyethersulfone, phenol formaldehyde resin, unsaturated polyester resin, epoxy resins, silicone resins, melamine resins, or urea formaldehyde.

In one embodiment, the volume of the first culture well is between 0.2-2 mL, the volume of the second culture well is between 0.2-2 mL.

In one embodiment, the sensing part further comprises a sensing electrode, configured to electrically connect to a sensor, wherein the sensor comprises electrochemistry sensor or semiconductor sensor.

In one embodiment, the circulating part further comprises a first input port and a second input port, arranged on the top surface respectively.

In one embodiment, the circulating part further comprises a first circulating unit and a second circulating unit, arranged between the first output port and the second output port, wherein the first circulating unit is fluidly connected with the first culture well, wherein the second circulating unit is fluidly connected with the second culture well.

A tissue culture system is provided, comprising: at least one or one or more tissue culture chips mentioned above, wherein one or more, or for example, two of tissue culture chips are fluidly connected.

A method for testing a tissue by using a tissue culture chip, comprising: (S1) culturing step: implanting a first tissue at the first culture well, adding a first culture medium to the first culture well, and (S2) bending step: bending the tissue culture chip, so that allows the first tissue become a second tissue.

In one embodiment, the (S2) bending step is started after the first tissue has cultured at the first culture well for a first time.

In one embodiment, the method further comprises (S3) assessing step, which is conducted after the bending step, comprising: assessing the second tissue with an RNA-related assay, assessing the second tissue with an antibody-related assay, assessing the first culture medium or assessing the second culture medium.

The beneficial effects of the present invention including:
(1) The tissue culture chip is applicable for culturing a tissue comprising ex vivo tissue.
(2) With the configuration of the sensing part, the tissue culture chip provides a platform beneficial for a study of the interaction of multiple tissues.
(3) The tissue culture chip, when applied to a drug test, enables monitoring of drug-assay-related parameters generated in the culturing part.
(4) The tissue culture chip provides complex or multiple culture environment changes formed in the first culture well or the second culture well by stretching or bending the flexible substrate, enabling observation of the cultured cells or tissues under changes of stress, for instance. Conceivably, the tissue culture chip allows simulations of heart beat, muscular contraction, or joint bending in a human body.
(5) The tissue culture system, established by integrating one or more tissue culture chips, can efficiently and properly simulate human physiology, thereby having more proper effects on drug test, for example; furthermore, the tissue culture chips can be easily reorganized, which allows any changes of the scale or the procedure of a test according to the demands.

### BRIEF DESCRIPTION OF THE DRAWINGS

FIG. 1 is a first schematic diagram of the tissue culture chip in one embodiment of the present invention.

FIG. 2 is a second schematic diagram of the tissue culture chip in one embodiment of the present invention.

FIG. 3 is a third schematic diagram in the tissue culture chip of one embodiment of the present invention.

FIG. 4 is a fourth schematic diagram of the tissue culture chip in one embodiment of the present invention.

FIG. 5 is a schematic diagram of the tissue culture system in one embodiment of the present invention.

FIG. 6 is a fifth schematic diagram of the tissue culture chip in one embodiment of the present invention.

FIG. 7 is flowchart of a tissue-testing method in one embodiment of the present invention.

FIG. 8 is a culture diagram of fibrosarcoma cells by using the tissue culture chip in one embodiment of the present invention.

FIG. 9 is a microscopic diagram of a fibrosarcoma tissue for presenting the structure thereof in one embodiment of the present invention.

### DETAILED DESCRIPTION OF THE INVENTION

The technical characteristics of the present invention will be described in detail hereafter by means of some embodiments with the drawings.

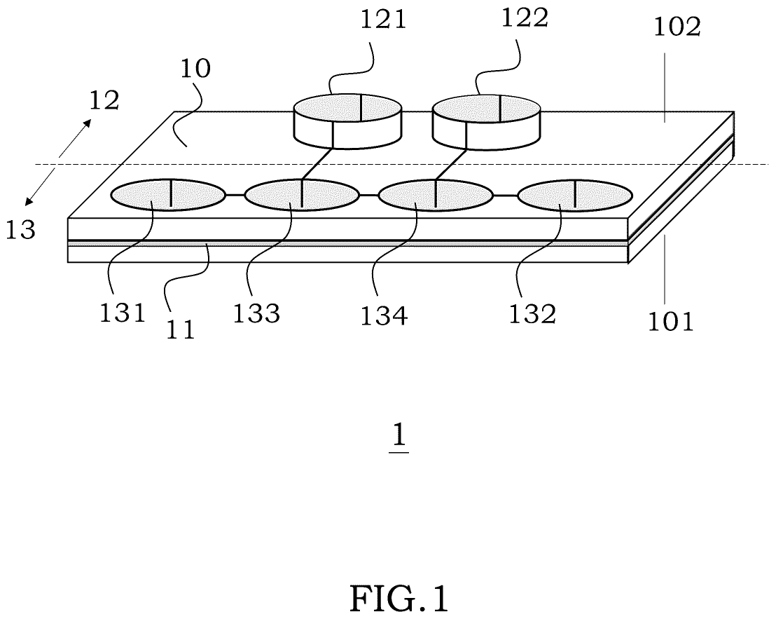

With reference to FIG. 1, FIG. 1 is a first schematic diagram of the tissue culture chip; in some embodiments, FIG. 1 illustrates a tissue culture chip 1 comprising: a flexible substrate 10, having a bottom surface 101 and a top surface 102, including: a sensing part 11, arranged between the bottom surface 101 and the top surface 102; a culturing part 12, arranged on the sensing part 11, comprising a first culture well 121 and a second culture well 122; and a circulating part 13, arranged on the sensing part and arranged near the culturing part, and fluidly connected to the culturing part 12.

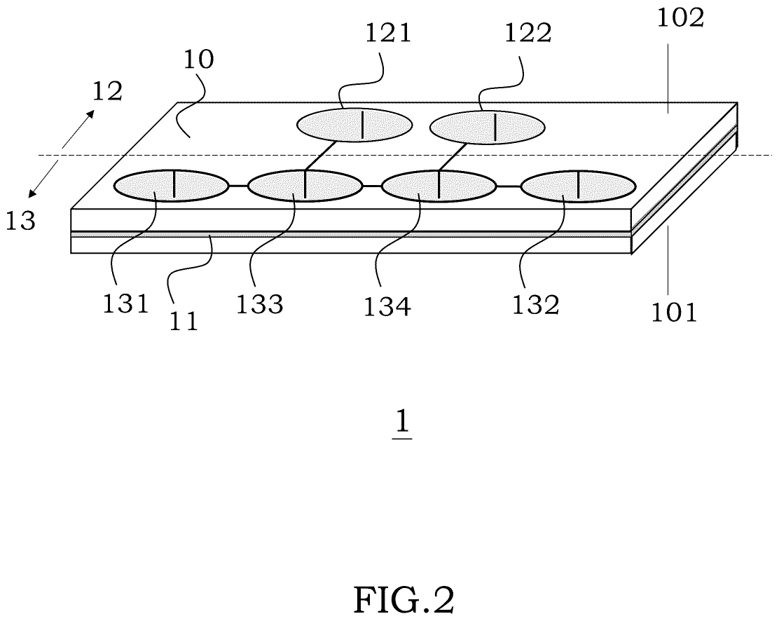

With reference to FIG. 1 and FIG. 2, FIG. 2 is a second schematic diagram of the tissue culture chip; in some embodiments, the first culture well 121 having a first opening, the second culture well having a second opening, the first opening and the second opening are arranged on the top surface 102 respectively, wherein the structure of the first opening and the structure of the second opening may be higher than the top surface 102 or share the same height with the top surface 102.

In some embodiments, the flexible substrate 10 has a length and a width, the length ranges from 50 to 150 millimeters, the width ranges from 20 to 100 millimeters.

In some embodiments, the flexible substrate 10 has a length and a width, the length ranges from 76.2 to 125 millimeters, the width ranges from 25.4 to 85 millimeters, and the length is greater than the width.

In some embodiments, the flexible substrate 10 having an end and the other end that is relatively located at the direction of the length; when the flexible substrate 10 is bent by getting the end and the other end of the flexible substrate 10 closer to each other toward the bottom surface 101 or toward the top surface 102, and: when a radius of curvature of the flexible substrate 10 is less than 50 (millimeter, mm), a Young's modulus of the flexible substrate 10 is less than 800 (megapascal, Mpa); when the radius of curvature of the flexible substrate 10 ranges between 50-100 (millimeter, mm), the Young's modulus of the flexible substrate 10 ranges between 800-1800 (megapascal, Mpa); when the radius of curvature of the flexible substrate 10 ranges between 80-200 (millimeter, mm), the Young's modulus of the flexible substrate 10 ranges between 1800-3000 (megapascal, Mpa).

In some embodiments, the first culture well 121 and the second culture well 122 disposed on the flexible substrate 10 along with the direction of the length.

In some embodiments, a material of the flexible substrate 10 is chosen from a group consisting of the following, but not limited to: polydimethylsiloxane, polyimide, polyethylene, poly (methyl methacrylate), polycarbonate, polyethylene terephthalate, polyethylene naphthalate, polytetrafluoroethylene, polyethersulfone, phenol formaldehyde resin, unsaturated polyester resin, epoxy resin, silicone resin, melamine resin, urea formaldehyde or any combination thereof.

In some embodiments, the flexible substrate 10 is transparent, partially transparent, or opaque.

In some embodiments, the surface of the flexible substrate 10, the surface of the first culture well 121, or the surface of the second culture well 122 is coated with a high dielectric constant material via sol-gel process, wherein the high dielectric constant material comprises hafnium oxide (HfO2).

In some embodiments, the surface of the flexible substrate 10, the surface of the first culture well 121, or the surface of the second culture well 122 is processed by oxygen plasma treatment, wherein the surface of the flexible substrate 10, the surface of the first culture well 121, or the surface of the second culture well 122 has the high dielectric constant material.

In some embodiments, the volume of the first culture well 121 ranges between 0.1-4 milliliter (mL), comprising: 0.1 mL, 0.2 mL, 0.3 mL, 0.4 mL, 0.5 mL, 0.6 mL, 0.7 mL, 0.8 mL, 0.9 mL, 1.0 mL, 2.0 mL, 3.0 mL, or 4.0 mL; the volume of the second culture well 122 ranges between 0.1 mL, 0.2 mL, 0.3 mL, 0.4 mL, 0.5 mL, 0.6 mL, 0.7 mL, 0.8 mL, 0.9 mL, 1.0 mL, 2.0 mL, 3.0 mL, or 4.0 mL; preferably, the volume of the first culture well 121 and the volume of the second culture well 122 ranges between 0.2-2 mL.

In some embodiments, the volume of the first culture well 121 is equal to the volume of the second culture well 122, or the volume of the first culture well 121 is not equal to the volume of the second culture well 122.

In some embodiments, the first culture well 121 or the second culture well 122 is used for culturing a tissue or a cell, wherein the tissue comprises suspension tissue or adherent tissue, wherein the cell comprises suspension cell or adherent cell; preferably the tissue is adherent tissue, the cell is adherent cell.

In some embodiments, the tissue or the cell adheres to any side of the first culture well 121 or the second culture well 122; preferably, the tissue or the cell adheres to the bottom of the first culture well 121 or the second culture well 122.

In some embodiments, the tissue culture chip 1 further comprises a flexible lid, which is arranged on the top surface 102 optionally.

In some embodiments, the circulating part 13 further comprises a first input port 131 and a first output port 132, arranged on the top surface 102 respectively.

In some embodiments, the flexible substrate 10 has a first lateral surface 103 and a second lateral surface 104, and the first lateral surface 103 and the second lateral surface 104 are arranged on opposite sides of the flexible substrate 10 respectively, wherein the first input port 131 is arranged on the first lateral surface 103, wherein the first output port 132 is arranged on the second lateral surface 104.

In some embodiments, the first input port 131 is structurally complementary to the first output port 132, so that the first input port 131 can be directly connected to the first input port 132.

In some embodiments, the circulating part 13 further comprises a first circulating unit 133 and a second circulating unit 134, both of them are arranged between the first input port 131 and the first output port 132, wherein the first circulating unit 133 is fluidly connected to the first culture well 121, wherein the second circulating unit 134 is fluidly connected to the second culture well 122.

In some embodiments, the first circulating unit 133 is fluidly connected to the first input port 131, the second circulating unit 134 is fluidly connected to the first output port 132, and the first circulating unit 133 is fluidly connected to the second circulating unit 134.

With regard to FIG. 1, in some embodiments, by filling any kind of fluid, the establishment of fluid connection between the first culture well 121 and the first circulating unit 133 can be achieved based on the principle of communicating vessels, and the establishment of fluid connection between the second culture well 122 and second circulating unit 134 is the same.

In some embodiments, any kind of fluid can flow between the circulating part 13 and the culturing part 12, and the any kind of fluid may comprise: culture medium, drug, culture medium containing drug, buffered saline, or buffered saline containing drug.

With regard to FIG. 1, in some embodiments, the first input port 131 is configured to allow the culture medium to flow in, the first output port 132 is configured to allow the culture medium to flow out, so that the culture medium can flow through the first input port 131, the first circulating unit 133, the second circulating unit 134 and the first output port 132 in sequence; further, the first circulating unit 133 is fluidly connected to the first culture well 121, the second circulating unit 134 is fluidly connected to the second culture well 122, which allows an exchange of substances to occur between the first culture well 121 and the first circulating unit 133, or to occur between the second culture well 122 and the second circulating unit 134, wherein the substances include nutrition or waste so that the growth of cells or tissue in the first culture well 121 or the second culture well 122 can be supported.

With regard to FIG. 1, in some embodiments, the first input port 131 is configured to allow a medium containing a drug to flow in, and followed by flowing through the first culture well 121 and the second culture well 122, then, the medium containing the drug that has flowed through the first culture well 121 and the second culture well 122 can be collected from the first output port 132, wherein the medium containing the drug may further comprises components produced by living cells or tissue cultured in the first culture well 121 or the second culture well 122.

With regard to FIG. 1, in some embodiments, the first culture well 121 is fluidly connected to the second culture well 122, which, comprehensibly, increases the interaction between the first culture well 121 and the second culture well 122.

With regard to FIG. 1, in some embodiments, the connection of fluid between units of the culturing part 12, units of the circulating part 13, or between units of the culturing part 12 and units of the circulating part 13, is adjustable; in other words, whether the fluid connection is established between the first culture well 121, the second culture well 122, the first circulating unit 133, or the second circulating unit 134, is not limited.

With regard to FIG. 1, in some embodiments, for example, the flow volume of culture medium between the first culture well 121 and the first circulating unit 133 can be more than, equal to, or less than the flow volume of culture medium between the second culture well 121 and the second circulating unit 134; in other words, the flow volume provided by the configuration is adjustable and not limited to this.

With regard to FIG. 1, in some embodiments, for example, the flow direction provided by the configuration between the first culture well 121 and the first circulating unit 133 that is relative to the bottom surface 101, or the flow direction provided by the configuration between the first circulating unit 133 and the second circulating unit 134 that is relative to the bottom surface 101 forms different angles with the bottom surface 101; in other words, the angle of the flow direction of fluids provided by the configuration is adjustable and not limited to this.

In some embodiments, the sensing part 11 further comprises a sensing electrode that is electrically connected to a sensor, wherein the sensor comprises an electrochemistry sensor or a semiconductor sensor.

With reference to the table 1, table 1 presents sensing items and the sensing range thereof that provided by the sensor; in some embodiments, a qualitative analysis of the fluids can be done by using the sensor, thereby obtaining a data; for example, the data may indicate components of the fluids comprising: organic components, gaseous components, inorganic components, ionic components or a concentration of any one of the aforesaid components.

TABLE 1

| Sensing items | Sensing range | Unit |
|---|---|---|
| pH value | 5-11 | |
| Partial pressure of tissue oxygen | 0-200 | mmHg |
| (Carbon dioxide) CO₂ | 0-5000 | ppm |
| (Nitric oxide) NO | 1-100 | μM |
| (Hydrogen peroxide) H₂O₂ | 1-20 | μM |
| (Reactive oxygen species) ROS | 10⁻⁷-10⁻³ | μM |
| (Adenosine triphosphate) ATP | 10⁻⁶-10 | μM |
| Glutamate | 0.02-0.3 | μM |
| Glucose | 0.5-15 | μM |
| Choline | 0.05-0.35 | μM |
| Ethanol | 0.1-0.8 | μM |
| (Xanthine Oxidase) XO | 15.6-500 | mU/mL |

In some embodiments, based on the flexibility of flexible substrate 10, there are at least two methods that can be used for influencing the growth of the cells inside the culturing part 12:

(1) By means of an inflation method comprising inflating an air cavity part 14 arranged inside the flexible substrate 10, and then the air cavity part 14 compresses the culturing part 12.

(2) By means of a force exerting method comprising exerting a force to bend, tense, shear or twist the flexible substrate 10, and the culturing part 12 can be affected.

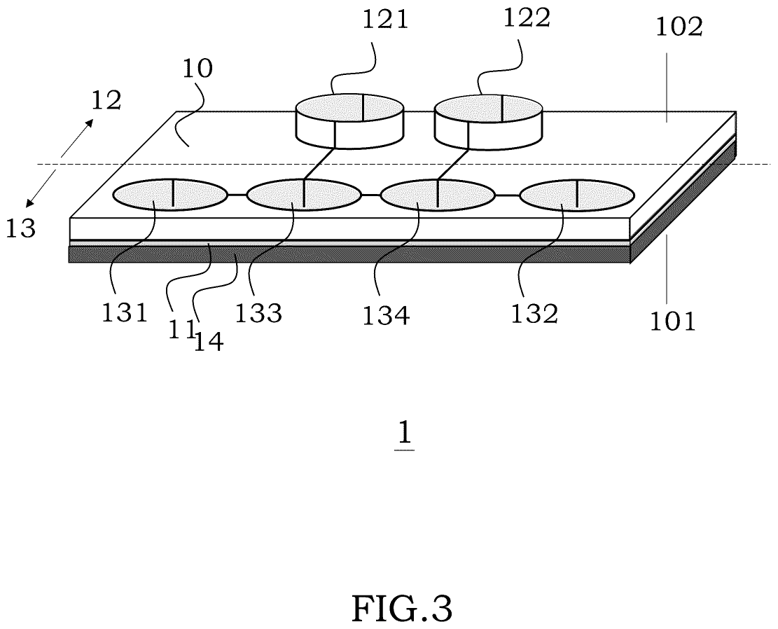

With regard to FIG. 3, FIG. 3 is a third schematic diagram of the tissue culture chip; in some embodiments, the flexible substrate 10 further comprises the air cavity part 14 formed on a side of the culturing part 12.

In some embodiments, the air cavity part 14 is arranged between the culturing part 12 and the sensing part 11, when the air cavity part 14 is inflated, the air cavity part 14 will become larger, thereby bulging the bottom of the first culture well 121 or bulging the bottom of the second culture well 122.

In some embodiments, the air cavity part 14 is arranged between the bottom surface 101 and the sensing part 11, when the air cavity part 14 is inflated, the air cavity part 14 will become larger, thereby bulging the bottom of the first culture well 121 or bulging the bottom of the second culture well 122 via the sensing part 11.

In some embodiments, the air cavity part 14 makes the bottom of the first culture well 121 or the bottom of the second culture well 122 bulge, or the air cavity part 14 makes the bottom of the first culture well 121 and the bottom the second culture well 122 bulge at the same time.

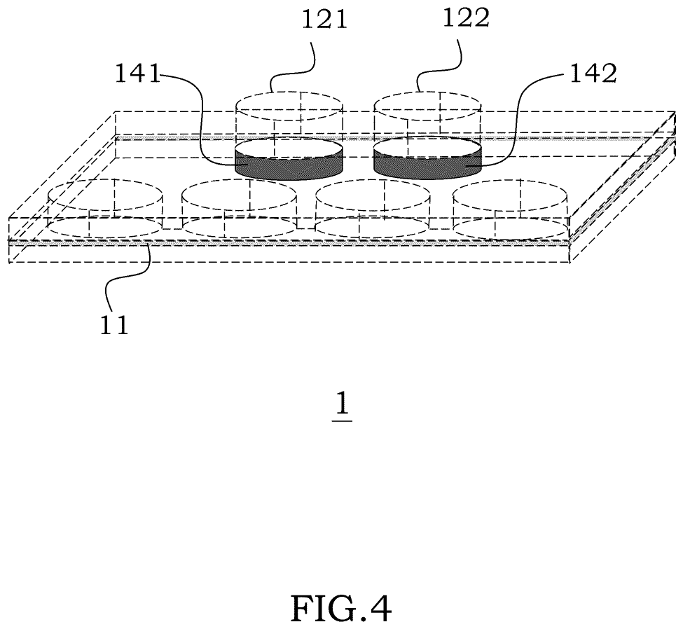

With regard to FIG. 4, FIG. 4 is a fourth schematic diagram of the tissue culture chip; in some embodiments, the air cavity part 14 further comprises a first cavity 141 and a second cavity 142, wherein the first cavity 141 corresponds to the first culture well 121, the second cavity 142 corresponds to the second culture well 122, the first cavity 141 and the second cavity 142 are configured to make both of the bottom of the first culture well 121 and the bottom of the second culture well 122 bulge.

Reasonably, being more specific, the inflation level of the first cavity 141 and the inflation level of the second cavity 142 are the same or not the same.

With regard to FIG. 4, both of the bottom of the first culture well 121 and the bottom of the second culture well 122 can be depressed by deflating the first cavity 141 and the second cavity 142 respectively.

Therefore, it is further realized that the first cavity 141 and the second cavity 142 can be inflated or deflated to affect the first culture well 121 and the second culture well 122.

In some embodiments, the first cavity 141 further comprises a first air orifice, and the second cavity 142 further comprises a second air orifice, wherein the first air orifice is arranged on a random side of the flexible substrate 10, and the second air orifice is arranged on another random side of the flexible substrate 10.

In some embodiments, by means of a force exerting method, the flexible substrate 10 is twisted or bent to change, to test or to stimulate a cell on a culture condition inside the culturing part 12, wherein the force exerting method may have different parameters including force direction, force level or force frequency.

Said force exerting method or the ‘twist’ of the flexible substrate 10 is not limited, for example, the force exerting method can comprise: a first force exerting method, or a second force exerting method.

The first force exerting method provides a consistent force that allows the flexible substrate 10 to be maintained in a first warping configuration.

The second force exerting method provides an intermittent force that allows the flexible substrate 10 to switch between the first warping configuration and a second warping configuration, wherein, the frequency of intermittent force is not limited.

In addition, it is not limited that the force exerting method further comprises a third force exerting method, or a third warping configuration or a fourth warping configuration provided by any of the force exerting method mentioned above.

To be clear, for example, the first force exerting method may be used for simulating a part of a human body receiving a consistent bending or twisting force; the second force exerting method may be used for simulating a part of a human body receiving an intermittent bending or twisting force.

Obviously, it could be free to combine the aforesaid methods comprising the first force exerting method or the second force exerting method, to change the shape of the flexible substrate 10.

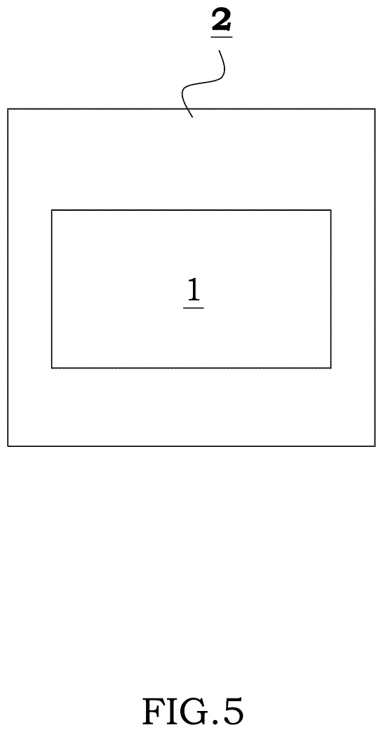

With regard to FIG. 5, FIG. 5 is a schematic diagram of the tissue culture system; the present invention further provides a tissue culture system 2 comprising at least one or plurality of the tissue culture chips 1, wherein the plurality of the tissue culture chips 1 are fluidly connected to each one of the tissue culture chips 1.

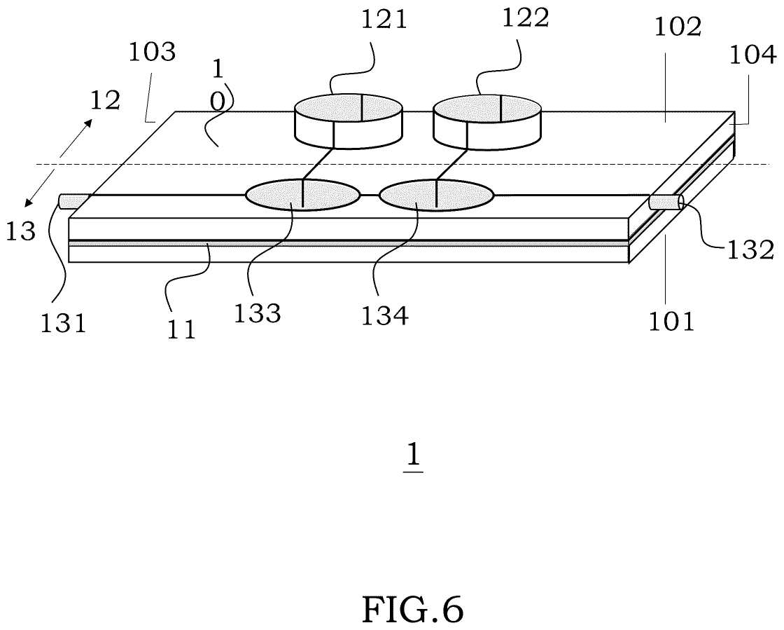

With regard to FIG. 6, FIG. 6 is a fifth schematic diagram of the tissue culture chip; in some embodiments, the plurality of the tissue culture chips 1 are connected to each other via the first input port 131 and the first output port 132 to form the fluid connection.

With regard to FIG. 6, in some embodiments, defining that the flexible substrate 10 has a first lateral surface 103 and a second lateral surface 104, the first lateral surface 103 is arranged correspondingly to the second lateral surface 104, the first input port 131 is arranged on the first lateral surface 103, and the first output port 132 is arranged on the second lateral surface 104; preferably, the distance between the first input port 131 and the bottom surface 101 is identical to the distance between the first output port 132 and the bottom surface 101.

In some embodiments, the tissue culture system 2 further comprises a connecting unit which can be optionally arranged between the plurality of tissue culture chips 1 to establish their connection.

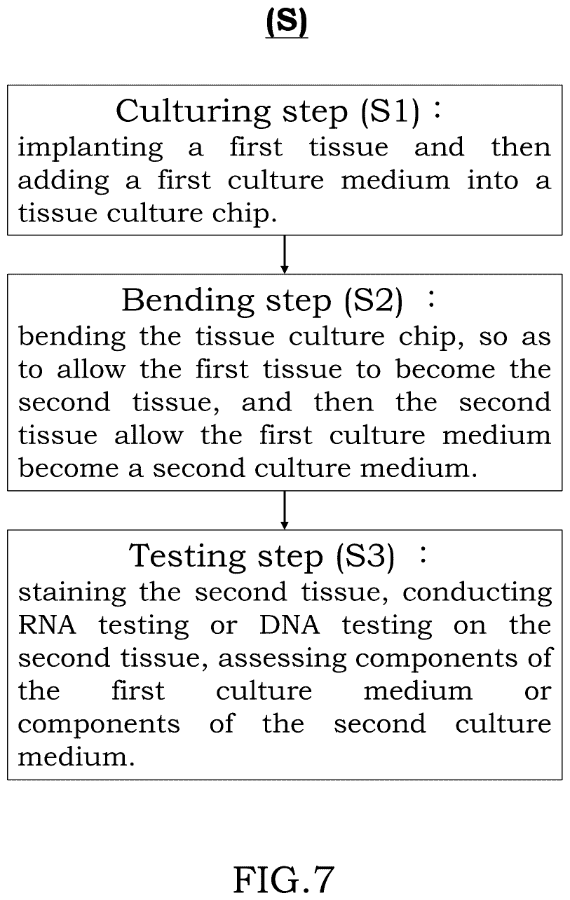

With regard to FIG. 7, FIG. 7 is a tissue-testing flow diagram of the tissue culture chip; in some embodiments, the present invention further discloses a tissue testing method (S), comprising a culturing step (S1): implanting a first tissue and then adding a first culture medium into said tissue culture chip 1; and a bending step (S2): bending the tissue culture chip 1, so as to allow the first tissue to become the second tissue, and then the second tissue allows the first culture medium become a second culture medium.

Notably, the first tissue can be identical or not identical to the second tissue; for example, when the first tissue has a stem cell property comprising differentiation ability, it should have possibility that the first tissue may change inherently after being cultured for a period of time.

The culture step (S1) includes: implanting the first tissue into the culturing part 12, or specifically, implanting the first tissue into the first culture well 121 or the second culture well 122; it is understood that there is no reason to limit the cell number of the first tissue.

The culture step (S1) includes: making the first culture medium circulate in the circulating part 13, or specifically, making the first culture medium circulate in the first circulating unit 133 or the second circulating unit 134.

The bending step (S2) begins after the first tissue has been cultured for a first time in the tissue culture chip 1; concretely, the bending step (S2) begins after the first tissue has reached a stable status, wherein the stable status comprises the first tissue attaching to the first culture well 121 or to the second culture well 122, or the first tissue starting to grow or to divide, and the first time is the time for the first tissue to reach the stable status; therefore, the first time is not limited, it could be minutes, hours or even days.

The bending step (S2) comprising: getting both sides of the flexible substrate 10 closer to each other toward the bottom surface 101 or toward the top surface 102, and there are multiple ways to bend the flexible substrate 10 but not limited to this.

With regard to FIG. 7, the tissue testing method (S) further comprises a testing step (S3), comprising: staining the second tissue, conducting RNA testing or DNA testing on the second tissue, assessing components of the first culture medium or components of the second culture medium.

The culture step (S1) includes: adding an additive into the circulating part 13, wherein the additive comprises: nutrients or drugs.

The tissue testing method (S) further comprises a (S0) step before the culture step (S1), including: preprocessing the first tissue, wherein the preprocessing step (S0) comprises: enzyme process, or slicing process, wherein the first tissue is an artificially obtained tissue (in vitro cultured tissue) or an ex vivo tissue.

The following provides a first embodiment of the present invention, which comprises culturing a cell using the tissue culture chip 1.

Wherein the cell comprising: fibrosarcoma cell, fibroadenoma cell, or melanoma cell.

Wherein the cell is cultured in a culture medium comprising: DMEM, EMEM, MEM, IMDM, RPMI-1640, or F-12, wherein those culture media could be further supplemented with fetal bovine serum (FBS), pyruvate, antibiotics, 2-mercaptoethanol, or HEPES.

A pump is used to transfuse the culture medium into the tissue culture chip 1, wherein the flow rate of the transfusion is between 1 μL/min to 120 μL/min, and 10 μL/min may be better; the temperature of the culture medium is controlled to be between 35-40° C., and better is 37° C.

A culture well described hereinafter can be the first culture well 121 or the second culture well 122 as mentioned above.

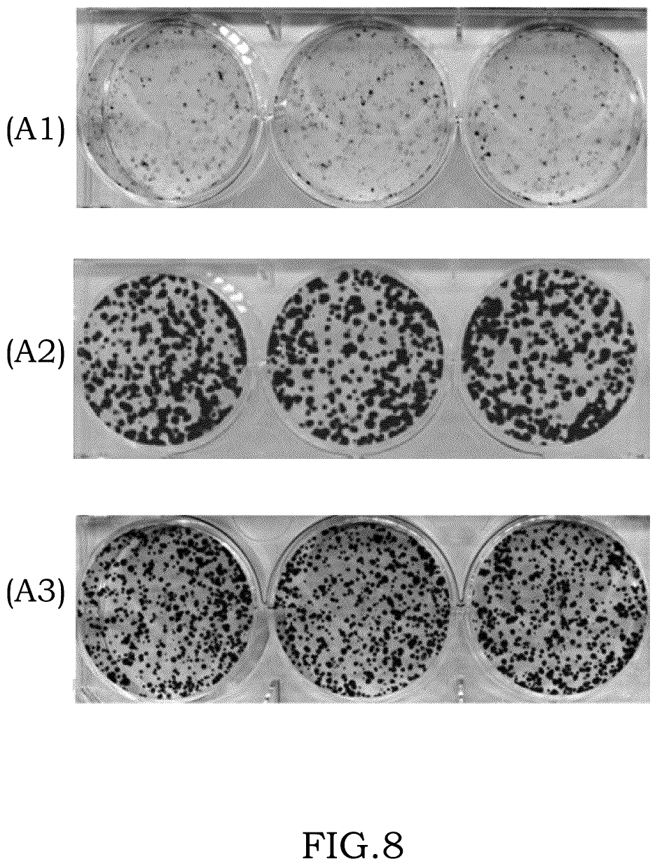

With regard to FIG. 8, FIG. 8 is a culture diagram of fibrosarcoma cells by using the tissue culture chip; 2000 of the fibrosarcoma cells were cultured in the culture well, and those fibrosarcoma cells then were stained for observation after culturing for 3 days (A1), 5 days (A2), and 8 days (A3), wherein (A1), (A2), and (A3) represent a triplicate test; obviously, as shown in FIG. 8, the number of and the total volume of the fibrosarcoma cells were both increased. In other words, the tissue culture chip 1 can sustain the growth of a cell or a tissue.

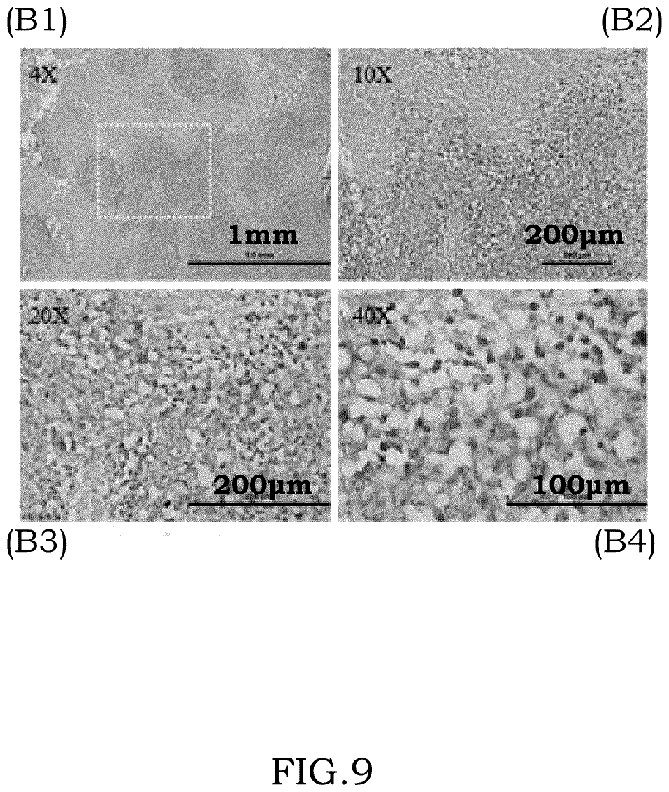

With regard to FIG. 9, FIG. 9 is a microscopic diagram of fibrosarcoma tissue and the structure thereof; each of the fibrosarcoma cell becomes a fibrosarcoma tissue after culturing by using the tissue culture chip 1 for 3 days. Subsequently by means of hematoxylin and eosin staining method, microscopic observations at 4-times magnification (B1), 10-times magnification (B2), 20-times magnification (B3), and 40-times magnification (B4) were taken respectively, wherein the scale of magnification of each is 1 mm (B1), 200 μm (B2), 200 μm (B3), and 100 μm (B4); as shown in FIG. 9, the fibrosarcoma cells become the fibrosarcoma tissue after culturing at the tissue culture chip 1, wherein the fibrosarcoma tissue demonstrated its tissue structural features therein.

The following provides a second embodiment comprising: culturing fibrosarcoma cells using the tissue culture chip 1, wherein the cells are further stimulated by bending the tissue culture chip 1 or not.

The first example: bending the tissue culture chip 1 after culturing the fibrosarcoma cells for 48 hours, wherein the bending means gets both sides of the flexible substrate 10 closer to each other toward the bottom surface 101 or toward the top surface 102, wherein the bending changes every 3 hours and a radius of curvature of the tissue culture chip 1 variates between 0 mm, 1000 mm, and 500 mm.

The first comparative example: culturing the fibrosarcoma cells without bending the tissue culture chip 1.

The second comparative example: bending the tissue culture chip 1 after culturing the fibrosarcoma cells for 48 hours, wherein the bending means getting both sides of the flexible substrate 10 closer to each other toward the bottom surface 101 or toward the top surface 102, wherein the bending changes every 3 hours and a radius of curvature of the tissue culture chip 1 variates between 0 mm, 1000 mm, and 100 mm.

With regard to Table 2, Table 2 demonstrates results of the fibrosarcoma cell numbers; it could be inferred from Table 2 that: (1) regarding the first example, bending the tissue culture chip 1 increased the growth of the fibrosarcoma cells, given the frequency of bending or the changing of the radius of curvature of the tissue culture chip 1 being appropriate; (2) regarding the first comparative example, the cell number of the fibrosarcoma in the first comparative example is less than in the first example; (3) regarding the second comparative example, compared to the first example, bending the tissue culture chip 1 did not increase the growth of the fibrosarcoma cells, given the frequency of bending or the changing of the radius of curvature of the tissue culture chip 1 being inappropriate, especially when the radius of curvature was excessively small.

TABLE 2

| | | The first | The second |
|---|---|---|---|
| | The first | comparative | comparative |
| | example | example | example |
| Culturing for 0 day | 1 × 10³ | 2 × 10³ | 1 × 10³ |
| Culturing for 3 days | 5 × 10⁴ | 3 × 10⁴ | 3 × 10⁴ |
| Culturing for 5 days | 6 × 10⁵ | 5 × 10⁵ | 3 × 10⁵ |
| Culturing for 8 days | 9 × 10⁷ | 6 × 10⁶ | 3 × 10⁶ |

Accordingly, the present invention discloses a tissue culture chip 1, based on the flexibility of the flexible substrate 10, a cell can be mechanically stimulated when cultured in the culturing part 12, and then the growth of the cell will be changed.

The following discloses a third embodiment of the present invention, comprising connecting the tissue culture chips 1 in parallel or in series to establish a tissue culture system 2, the tissue culture system 2 allows a cell to be cultured for a long period of time or to be conducted with the aforesaid bending tests in the tissue culture chip 1.

The tissue culture system 2 further comprises a pump for driving the culture medium.

The tissue culture system 2 further comprises a temperature controller for controlling the temperature of the culture medium.

The tissue culture system 2 further comprises a bending device for bending the tissue culture chip 1.

According to the embodiments and the examples described therein, in the first aspect, by means of the tissue culture chip 1 provided by the present invention, a cell grows into a tissue with tissue-like structure; in the second aspect, by means of the tissue culture chip 1 provided by the present invention, by bending the flexible substrate 10, a cell or a tissue grows in the tissue culture chip 1 can be stimulated, and then physiology of the cell or the tissue may also be changed, wherein the physiology includes one or more of the following: growth amount, cytokine production, or cell differentiation.

In the third aspect, the tissue culture chip 1 is part of the tissue culture system 2, wherein the tissue culture system 2 supports transfusion of the culture medium into the tissue culture chip 1, and adjustment of the temperature or the flow rate of the culture medium. The tissue culture system 2 may include a bending device so as to bend the tissue culture chip 1.

The disclosure has been described above are just some preferred embodiments of the present invention, and should not be used for limiting the claims of the present invention; In other words, any modifications and similar arrangements based on the present invention, should be included and protected by the claim of the present invention.

1. A tissue culture chip, comprising:
a flexible substrate, having a bottom surface and a top surface, including:
a sensing part, arranged between the bottom surface and the top surface;
a culturing part, arranged on the sensing part, comprising a first culture well and a second culture well; and
a circulating part, arranged on the sensing part and arranged near the culturing part.

2. The tissue culture chip of claim 1, wherein a Young's modulus of the flexible substrate ranges between 800-1800 (megapascal, Mpa), when the flexible substrate is being bent to enable both end of the flexible substrate getting closer toward the bottom surface or the top surface and thereby a radius of curvature of the flexible substrate ranges between 50-100 (millimeter, mm).

3. The tissue culture chip of claim 1, wherein a material of the flexible substrate is chosen from a group consisting of the following: polydimethylsiloxane, polyimide, polyethylene, poly (methyl methacrylate), polycarbonate, polyethylene terephthalate, polyethylene naphthalate, polytetrafluoroethylene, polyethersulfone, phenol formaldehyde resin, unsaturated polyester resin, epoxy resin, silicone resin, melamine resin, urea formaldehyde or any combination thereof.

4. The tissue culture chip of claim 1, wherein the sensing part further comprises a sensing electrode, configured to electrically connect to a sensor.

5. The tissue culture chip of claim 1, wherein the circulating part further comprises a first input port, arranged on the top surface.

6. The tissue culture chip of claim 12, wherein the circulating part further comprises a first circulating unit, arranged between the first input port and the first output port.

7. A tissue culture system, comprising:
at least one or plurality of the tissue culture chips, comprising:
a flexible substrate, having a bottom surface and a top surface, including:
a sensing part, arranged between the bottom surface and the top surface;
a culturing part, arranged on the sensing part, the culturing part comprising a first culture well and a second culture well; and
a circulating part, arranged on the sensing part and arranged near the culturing part, the circulating part is fluidly connected to the culturing part;
wherein the plurality of the tissue culture chips are fluidly interconnected.

8. A method for testing a tissue by using a tissue culture chip, comprising:
culturing step: implanting a first tissue into the tissue culture chip according to claim 1, then adding a first culture medium to the tissue culture chip for culturing the first tissue; and
bending step: bending the tissue culture chip, so as to allow the first tissue to become a second tissue, and then the second tissue allow the first culture medium to become a second culture medium.

9. The method of claim 8, wherein the bending step is started after the first tissue has been cultured at the tissue culture chip for a first time.

10. The method of claim 8, further comprising:
assessing step, comprising: staining the second tissue, assessing the second tissue using an RNA-related assay, assessing the second tissue using an antibody-related assay, assessing components of the first culture medium, or assessing components of the second culture medium.

11. The tissue culture chip of claim 4, wherein the sensor comprises electrochemistry sensor or semiconductor sensor.

12. The tissue culture chip of claim 5, wherein the circulating part further comprises a first output port, arranged on the top surface.

13. The tissue culture chip of claim 6, wherein the first circulating unit is fluidly connected to the first culture well.

14. The tissue culture chip of claim 6, wherein the circulating part further comprises a second circulating unit, arranged between the first input port and the first output port.

15. The tissue culture chip of claim 14, wherein the second circulating unit is fluidly connected to the second culture well.

16. The method of claim 8, wherein in the tissue culture chip, a Young's modulus of the flexible substrate ranges between 800-1800 (megapascal, Mpa), when the flexible substrate is being bent to enable both end of the flexible substrate getting closer toward the bottom surface or the top surface and thereby a radius of curvature of the flexible substrate ranges between 50-100 (millimeter, mm).

17. The method of claim 8, wherein in the tissue culture chip, a material of the flexible substrate is chosen from a group consisting of the following: polydimethylsiloxane, polyimide, polyethylene, poly (methyl methacrylate), polycarbonate, polyethylene terephthalate, polyethylene naphthalate, polytetrafluoroethylene, polyethersulfone, phenol formaldehyde resin, unsaturated polyester resin, epoxy resin, silicone resin, melamine resin, urea formaldehyde or any combination thereof.

18. The method of claim 8, wherein in the tissue culture chip, the sensing part further comprises a sensing electrode, configured to electrically connect to a sensor, the sensor comprises electrochemistry sensor or semiconductor sensor.

19. The method of claim 8, wherein in the tissue culture chip, the circulating part further comprises a first input port and a first output port, arranged on the top surface respectively.

20. The method of claim 19, wherein in the tissue culture chip, the circulating part further comprises a first circulating unit and a second circulating unit, arranged between the first input port and the first output port, wherein the first circulating unit is fluidly connected to the first culture well, the second circulating unit is fluidly connected to the second culture well.
